(19) United States
(12) Patent Application Publication
(10) Pub. No.: US 2004/0101483 A1
(43) Pub. Date: May 27, 2004

(54) Medical aerosol formulations

(76) Inventors: Rudi Muller-Walz, Schopfheim (DE); Carsten Niederlander, Riehen (CH)

Correspondence Address: EDWARDS & ANGELL, LLP, P.O. BOX 55874, BOSTON, MA, 02205, US

(21) Appl. No.: 10/473,874

(22) PCT Filed: Mar. 11, 2002
(86) PCT No.: PCT/CH02/00145

(30) Foreign Application Priority Data
Mar. 30, 2001 (CH) 60101
Aug. 20, 2001 (CH) 152701

(51) Int. Cl. A61L 9/04; A61K 9/14

(52) U.S. Cl. 424/46; 514/291

(57) ABSTRACT

Calcium salts, magnesium salts and zinc salts of palmitic acid and of stearic acid are suited for use as solid auxiliary agents for medical suspension aerosol formulations based on hydrofluoroalkanes. They improve, in particular, the suspension stability, the mechanical function of the dosing valve, the dosing precision, and the chemical stability of the active substance.

The present invention relates to medical suspension aerosol formulations and to the use of certain salts as excipients in such formulations.

For the production of medical metered-dose aerosols, as a rule only propellants which can be liquefied at room temperature are suitable. In the past, customary chlorofluorocarbons (CFCs), such as trichloromono-fluoromethane (F11), dichlorodifluoromethane (F12) and 1,2-dichloro-1,1,2,2-tetrafluoromethane (F114), and occasionally also short-chain alkanes, such as, for example, propane, butane and isobutane, were used.

On account of the ozone problem, caused by the cleavage of free-radical chlorine atoms from the CFCs, in the Montreal agreement many countries agreed no longer to use the CFC as propellants in the future. Suitable CFCs substitutes in the medical field are fluorinated alkanes, in particular hydrofluoroalkanes (in the context of the present invention also designated as “HFA”) such as 1,1,1,2-tetrafluoroethane (HFA 134a) and 1,1,1,2,3,3,3-heptafluoropropane (HFA 227), since they are inert and have a very low toxicity. On account of their physical properties, such as pressure, density etc., the latter are particularly suitable for replacing CFCs such as F11, F12 and F114 as propellants in metered-dose aerosols.

It is generally known that in the case of suspension formulations only active compound particles which are smaller than approximately 6 μm are able to enter the lungs. For the desired deposition of the active compounds in the lungs, these must therefore be pulverized or micronized before processing by means of special processes, such as, for example, pinned-disk, ball or air-jet mills. A grinding process, however, leads to a surface area enlargement, which as a rule is accompanied by an increase in the electrostatic charge of the micronized active compound, by means of which the flow behavior and the active compound dispersion is then usually impaired. As a result of the interfacial activities, agglomeration of active compound particles or alternatively the adsorption of active compounds on interfaces frequently occurs, which, for example, is evident in the accumulation on equipment or container surfaces.

In the case of aerosol preparations in which the active compound is present suspended in the liquefied propellant, deposition or ring formation can occur in the container at the site where the liquid phase changes into the gaseous phase. Without wetting the micronized active compound particles or conducting away the charges, or modifying their surface properties, suspensions can only be inadequately stabilized or kept in a dispersed state. The imperfect wetting or dispersion of the active compound particles also results in these in many cases having a high proneness to adsorption and adhering to surfaces such as the container inner wall or the valve, which leads to an underdosage and to a poor metering accuracy from spray burst (puff) to spray burst. A surface-active excipient must therefore as a rule be added to suspension formulations in order to lower the adsorption on interfaces and to achieve an acceptable metering accuracy. Alteration occurring in the course of storage is particularly problematical, in particular a lowering of the proportion of the inhalable particles which are able to enter the lungs, the “fine particle dose” (FPD), which leads to a decrease in the efficacy of the aerosol formulation.

To overcome these problems, as a rule permitted surface-active substances are added, as were already formerly used in CFC-containing formulations, and dissolved in the liquid phase. However, it has been shown that the customary excipients used in CFC-containing metered-dose aerosols, such as lecithin, sorbitan trioleate and oleic acid, are only inadequately soluble in hydrofluoroalkanes such as HFA 134 and HFA 227. In JP 55-361 B, CFC-containing aerosol formulations were also described which, as suspending excipient, contain a metal salt of a fatty acid, for example a calcium or aluminum stearate, magnesium oleate or zinc isostearate, together with an oil-soluble solvent, such as isostearic acid, 2-octyl-dodecanol, 2-hexadecanol, isopropyl myristate, trioleyl phosphate, diethylene glycol, diethyl ether and the like, in order to dissolve the metal salt. Such formulations, however, have not been successful in practice.

It was therefore proposed to leave out the surface-active excipients in HFA-containing formulations if possible or—if they are indispensable for technological reasons—to add a polar cosolvent such as, for example, ethanol in order to improve the solubility in a manner known per se and to dissolve the surface-active agents. Other solution proposals comprise coating the active compound particles with the surface-active agent or using special, propellant-soluble surface-active agents. Such proposals are found, for example, in U.S. Pat. No. 2,868,691, U.S. Pat. No. 3,014,844, DE-A-2 736 500, EP-A-0 372 777, WO-A-91/11495, EP-A-0 504 112, EP-B-0 550 031, WO-A-91/04011, EP-A-0 504 112 and WO-A-92/00061. In U.S. Pat. No. 5,676,931, it was proposed for formulations of LHRH analogs or 5-lipoxygenase inhibitors to add to the active compound/propellant mixture an excipient designated as a “protective colloid”, preferably cholesterol, sodium lauryl sulfate, stearic acid, caprylic acid or taurocholic acid. In WO-A-96/19198, pharmaceutical aerosol formulations were further described which, in addition to a propellant and an active compound suitable for inhalation, contain a surface-active agent, selected from C₈-C₁₆-fatty acids or salts thereof, bile acid salts, phospholipids and alkyl saccharides, and optionally up to 30% by weight of ethanol, bile acid salts being preferred and examples only being indicated for sodium taurocholate.

If cosolvents such as ethanol are added in higher concentrations, however, the density of the propellant mixture decreases, which can lead to undesired demixing, especially in the case of suspensions. Moreover, a “wet spray” can undesirably be obtained, because the propellant evaporates much more rapidly than ethanol. This is, inter alia, particularly disadvantageous, because at ethanol concentrations of, for example, 10% or more, on account of the completely different evaporation characteristics of ethanol to the propellant, particles having larger aerodynamic diameters are generated to an increased extent and the proportion of inhalable particles (<6 μm) decreases. As a result of this, a lowering of the fine particle dose (FPD) which is crucial for the efficacy occurs.

In addition, owing to the increase in the solubility during storage, partial solution effects can also occur, which leads to crystal growth and in turn to a lowering of the amount of inhalable particles which are able to enter the lungs, the “fine particle dose” (FPD). In the case of ethanol-containing aerosols, in addition problems of active compound stability can occasionally occur, in particular if the active compound is present in dissolved form.

This all might explain why most commercially available metered-dose aerosols were formulated as suspensions.

For the measurement of the aerodynamic particle size distribution or the FPD or the fine particle fraction (FPF), impactors are suitable, such as, for example, the 5-stage multistage liquid impinger (MSLI) or 8-stage Andersen cascade impactor (ACI), which are described in chapter <601> of the United States pharmacopeia (USP) or in the inhalant monograph of the European pharmacopeia (Ph. Eur.). With the aid of the aerodynamic particle distribution, it is possible by means of a “log-probability plot” (logarithmic representation of the probability distribution) to calculate the average aerodynamic particle diameter (mass median aerodynamic diameter MMAD) of the aerosol preparations. With this information for particle distribution, information is obtained on whether the active compound is more likely to be deposited in the upper or lower area of the lungs.

As follows from the foregoing, the maintenance of an adequately good metering accuracy, i.e. the constant release of active compound from spray burst to spray burst, is a fundamental problem of suspension metered-dose aerosols which is additionally complicated by the substitution of the CFCs. In addition to the valve and adapter, the metering accuracy depends essentially on the suspension properties, i.e. on how well and homogeneously the active compound is dispersed in the propellant and how long the suspension remains in this labile state of equilibrium without alteration of its physical properties. The maintenance of an acceptable metering accuracy proves to be particularly difficult in the case of potent, low-dose active compounds. For example, a formulation is needed for the long-acting beta-agonist formoterol fumarate, which is already active in very low doses (6 μg/stroke), which formulation affords an adequately stable suspension which does not adhere to interfaces and does not change in the course of storage under different temperature and moisture conditions. A general survey of the products available on the market shows that to date there is no metered-dose aerosol which can meter active compounds in amounts of less than 10 μg per stroke (i.e. per spray burst) with a scatter of better than ±25%.

The invention is therefore based on the object of as far as possible avoiding the problems of suspension metered-dose aerosols mentioned and making available medical suspension aerosol formulations which have improved suspension and keeping properties and make possible a good metering accuracy—even in the case of low-dose active compounds.

The object is achieved according to the invention by use of a carboxylic acid salt, selected from calcium, magnesium and zinc salts of palmitic and stearic acid, as a solid excipient in medical suspension aerosol formulations. It was in fact surprisingly found that these salts are suitable as suspending excipients for medical aerosol formulations, although they are poorly soluble in the customary propellants. Further, it was surprisingly found that these salts at the same time improve the valve function, i.e. act as valve lubricants. In this function, the salts mentioned cause a smoother, more frictionless actuation of the valves without excessive noise development and increase the metering accuracy. Surprisingly, it was furthermore found that they can also improve the chemical stability of the pharmaceutical active compound, in particular the moisture resistance of moisture-sensitive active compounds. The use of these salts thus makes possible the preparation of improved suspension aerosol formulations.

The invention therefore relates to the use of a carboxylic acid salt, selected from calcium, magnesium and zinc salts of palmitic and stearic acid, as a solid excipient in medical suspension aerosol formulations for inhalation, comprising a pressure-liquefied, non-toxic propellant of the general formula

CₓH_{y}F_{z} (I)

in which x is the number 1, 2 or 3, y and z are each an integer ≧1 and y+z=2x+2,

and a finely divided pharmaceutically active compound suspended in the propellant, and in particular the use of such a salt for improving the suspension stability of medical suspension aerosol formulations, for improving the metering accuracy of compressed gas packs of medical suspension aerosol formulations, for improving the valve function of the metering valve of pressurized gas packs and/or for improving the chemical stability, in particular the moisture resistance, of pharmaceutical active compounds in medical suspension aerosol formulations. The use of the palmitic and stearic acid salts utilizable according to the invention in aerosol formulations which contain a finely divided pharmaceutically active compound administrable by inhalation and, as a hydrofluoroalkane, (I) 1,1,1,2-tetrafluoroethane (HFA 134a) and/or 1,1,1,2,3,3,3-heptafluoropropane (HFA 227) is particularly advantageous. By this means—as described below—improved suspension aerosol formulations for active compounds such as formoterol, salmeterol, fenoterol, clenbuterol, levalbuterol, ipratropium, oxytropium, glycopyrronium, tiotropium, budesonide, ciclesonide, mometasone, fluticasone, beclomethasone, flunisolide, loteprednol, triamcinolone, amiloride, rofleponide, salbutamol, terbutaline and pharmaceutically acceptable salts and derivatives thereof can in particular be obtained.

The invention further relates to a medical aerosol formulation for inhalation, comprising a pressure-liquefied, nontoxic propellant of the general formula

CₓH_{y}F_{z} (I)

in which x is the number 1, 2 or 3, y and z are each an integer ≧1 and y+z=2x+2,

an efficacious amount of a finely divided pharmaceutically active compound suspended in the propellant and a solid excipient, selected from calcium, magnesium and zinc salts of palmitic and stearic acid. According to a preferred aspect, the invention relates in particular to a medical aerosol formulation, comprising

(a) a pressure-liquefied, nontoxic propellant, selected from 1,1,1,2-tetrafluoroethane, 1,1,1,2,3,3,3-hepta-fluoropropane and mixtures thereof,

(b) an efficacious amount of a finely divided pharmaceutically active compound suspended in the propellant, selected from formoterol, salmeterol, fenoterol, clenbuterol, levalbuterol, ipratropium, oxytropium, glycopyrronium, tiotropium, budesonide, ciclesonide, mometasone, fluticasone, beclomethasone, flunisolide, loteprednol, triamcinolone, amiloride, rofleponide, salbutamol, terbutaline and pharmaceutically acceptable salts and derivatives thereof, and

(c) a solid excipient, selected from calcium, magnesium and zinc salts of palmitic and stearic acid. The formulation is suitable in particular as a metered-dose aerosol for pressurized gas packs.

The invention furthermore relates to the preparation of the aerosol formulation according to the invention and to a pressurized gas pack comprising the aerosol formulation according to the invention in a pressure-safe container provided with a metering valve.

The calcium, magnesium and zinc salts of palmitic and stearic acid are soap-like compounds which are poorly soluble and as a rule are virtually insoluble in pressure-liquefied hydrofluoroalkanes or other propellants even with the addition of customary cosolvents such as ethanol. Surprisingly, it has been found, however, that the use of these salts in solid form facilitates the suspension of pharmaceutical active compounds in hydrofluoroalkanes and other propellants and that by this means medical metered-dose aerosols having improved quality-relevant properties, such as improved suspension stability, higher metering accuracy etc., can in particular be obtained. An oil-soluble solvent in order to dissolve the excipient in the formulation is not necessary and even undesirable according to the invention. This finding is all the more surprising as in GB-B 837 465 and U.S. Pat. No. 3,014,844 the use of dispersible surface-active excipients in CFC propellants was already discussed, but with respect to a blockage of the valve and adapter was assessed as unsuitable, and in JP 55-361 B an oil-soluble solvent had to be added in order to dissolve fatty acid salts.

If a pharmaceutically active compound, such as formoterol fumarate, levalbuterol sulfate and the like, is mixed with one of the suspending excipients utilizable according to the invention, a powder mixture is obtained which can be suspended readily in the customary propellants, as a rule also in the absence of dissolved surface-active agents. The suspensions obtained can moreover be accurately metered even in the case of very low-dose active compound concentrations, which could possibly be attributed to the formation of excipient-active compound associates. On account of these properties, the excipients utilizable according to the invention are therefore suitable, inter alia, for the improvement of the metering accuracy of suspension formulations and in particular as vehicles for the dilution of low-dose active compounds for the purpose of improving the metering accuracy.

In addition, it has been found that the proneness to adhesion of electrostatically charged active compounds is reduced by admixing the excipients utilizable according to the invention, by which means their dispersibility is improved.

Further, it has surprisingly been found that the use of the excipients utilizable according to the invention improves the mechanical function of the metering valves. Although these excipients are as a rule virtually insoluble in the propellants and are therefore present in suspended form, on account of their surface-active properties they apparently act as lubricants and thereby improve the valve function. The more uniform mechanical function of the valves leads as a result to a more consistent metering of the metered-dose aerosol to be administered and thus likewise to an improvement in the metering accuracy.

It has furthermore been found that the use of the excipients utilizable according to the invention improves the chemical stability, in particular the moisture resistance, of pharmaceutically active compounds present in the formulation, such as formoterol fumarate, formoterol tartrate, fenoterol hydrobromide, salbutamol sulfate, salbutamol acetate, levalbuterol sulfate, terbutaline sulfate, tiotropium bromide, budesonide, mometasone, fluticasone and the like, and and thus also the chemical stability of the aerosol formulation.

The excipients magnesium stearate, magnesium palmitate, calcium stearate, calcium palmitate, zinc stearate and zinc palmitate utilizable according to the invention therefore allow the preparation of improved suspension aerosol formulations and, if desired, the abandonment of the surface-active agents customarily used (oleic acid, sorbitan trioleate and lecithin), which are further utilizable in hydrofluoroalkanes only with use of a cosolvent. Suitable stearates utilizable according to the invention are in particular also commercially available stearates which can contain up to approximately one-third of corresponding palmitate. Magnesium stearate and mixtures of magnesium stearate and magnesium palmitate are particularly preferred.

The aerosol formulation according to the invention can contain the pharmaceutically active compound, if desired in the form of a pharmaceutically acceptable salt or derivative, such as, for example, formoterol fumarate, formoterol tartrate, salmeterol xinafoate, fenoterol hydrobromide, clenbuterol hydrochloride, levalbuterol sulfate, ipratropium bromide, oxytropium bromide, glycopyrronium bromide, tiotropium bromide, mometasone furoate, fluticasone dipropionate, beclomethasone dipropionate, flunisolide acetate, salbutamol sulfate, salbutamol acetate or terbutaline sulfate. Active compounds having chiral centers can be used in the form of their active enantiomer or as an enantiomer mixture (e.g. racemate). If desired, the aerosol formulations according to the invention can also contain two or more pharmaceutically active compounds, combinations of fluticasone, ipratropium, oxytropium, glycopyrronium, tiotropium, budesonide, mometasone, ciclesonide, rofleponide or a pharmaceutically acceptable salt or derivative thereof with salbutamol, levalbuterol, fenoterol, terbutaline, formoterol and/or salmeterol or a pharmaceutically acceptable salt or derivative thereof being preferred. If desired, the aerosol formulations according to the invention can also contain, in addition to one or more suspended active compounds, dissolved pharmaceutically active compounds.

The content of pharmaceutically active compound in the aerosol formulations according to the invention is not critical and is as a rule dependent especially on the desired, therapeutically or prophylactically active dose and thus on the activity of the respective active compound. For example, the content of suspended pharmaceutically active compound can be approximately 0.0001 to 5% by weight or more, preferably approximately 0.001 to 2% by weight, based on the total formulation. Since the advantages of the aerosol formulation according to the invention are particularly marked in the case of highly active, i.e. low-dose, active compounds, it is particularly suitable for formulations having comparatively low active compound concentrations of, for example, approximately 0.0001 to 0.4% by weight, 0.001 to 0.1% by weight or 0.001 to 0.04% by weight. Since the stroke masses of commercially available MDIs (metered dose inhalers) are mostly in the range from approximately 30 to 130 mg (with valves corresponding to approximately 25 to 100 μl) and typically approximately 70 mg, using the formulations according to the invention in particular also doses of approximately 0.1 to 100 μg, 0.1 to 50 μg or 0.1 to 20 μg of pharmaceutically active compound can be administered per spray burst.

The active compound to be suspended or the active compounds to be suspended can be obtained in a manner known per se, e.g. by means of pinned-disk, ball or air-jet mills, micronized or by controlled micro-crystallization or precipitation, and suspended in the propellant. In order to guarantee an inhalability which is as complete as possible and to avoid small particles being exhaled again, the suspended active compound particles preferably have a mean aerodynamic particle diameter MMAD (mass median aerodynamic diameter, mass average) in the range from approximately 1 to 6 μm, for example approximately 2 to 5 μm.

The excipients utilizable according to the invention are known to the person skilled in the art and are commercially obtainable or can be prepared from the carboxylic acids in a known manner; for example alkaline earth metal, aluminum and zinc salts of long-chain carboxylic acids are occasionally used as excipients in the preparation of water-in-oil emulsions. The expression “solid salt” or “solid excipient” in the context of the present invention in particular comprises those salts or excipients which can be present at 20° C. in crystalline or amorphous form, those which can still be present in crystalline or amorphous form at approximately 50° C. or 60° C. being preferred. Of course, excipients are also suitable which contain both crystalline and amorphous fractions. Suitable forms according to the invention—as mentioned above—are in particular also commercially available forms of the excipients, such as, for example, commercially available magnesium stearate, which can typically contain up to approximately one-third of magnesium palmitate.

The particle size of the excipient utilized according to the invention is not critical. If desired, the excipient can likewise be employed in micronized form having a mean aerodynamic particle diameter MMAD of approximately 1 to 6 μm, for example approximately 2 to 5 μm, in particular if the simultaneous inhalation of the excipient is desired. The micronization can be carried out in a manner known per se according to the methods mentioned above in connection with the active compound. However, excipient with a mean aerodynamic particle diameter MMAD of more than 6 μm, for example approximately 10 to 100 μm, is preferably used if it is desired that the excipient does not reach the lung.

The proportion of solid suspending excipient in the formulations according to the invention can vary within a relatively wide range, usually even small amounts being adequate in order to achieve the desired improvements. Typically, the weight ratio between the suspended pharmaceutically active compound and excipient can be approximately 50:1 to approximately 1:10, a range from approximately 10:1 to approximately 1:5 usually being preferred. Based on the total formulation, the proportion of solid excipient can typically be approximately 1% by weight or less, for example approximately 0.0001 to 1% by weight; higher amounts, however, are as a rule not disadvantageous. In general, however, amounts of approximately 0.005 to 0.5% by weight, in particular approximately 0.01 to 0.2% by weight, based. on the total formulation, are preferred, in particular if the active compound is likewise present in a low concentration. The excipient content per spray burst is therefore in general not more than approximately 500 μg and usually is in the range from approximately 5 to 250 μg or 10 to 100 μg.

Preferably, the excipient, depending on the active compound and propellant used, can be selected such that the density of the suspended materials is adjusted as far as possible overall to the density of the propellant. For example, micronized formoterol fumarate, which is prone to flotation in HFA 227, can be combined with magnesium stearate, which is prone to sedimentation, in order to keep the suspended material better in suspension and to minimize flotation or sedimentation, whereby the physical stability of the suspension is further improved.

HFA 134a and HFA 227 have a vapor pressure of about 6 bar and about 4.2 bar respectively at 20° C. These two propellants differ with respect to their density (about 1.2 g/ml for HFA 134a and about 1.4 g/ml for HFA 227), which is of importance insofar as by suitable choice of the propellant or propellant mixture its density can be adjusted better to the density of the suspended substances and thus the latter can be kept in suspension better. If desired, the density of the propellant can also be further lowered by addition of cosolvents or other propellants, such as, for example, ethanol, diethyl ether, propane, n-butane, isobutane and the like. In view of the ozone problem, however, preferably no or only small amounts of CFCs are used.

In the aerosol formulations according to the invention, the proportion of 1,1,1,2-tetrafluoroethane (HFA 134a) and/or 1,1,1,2,3,3,3-heptafluoropropane (HFA 227), based on the total formulation, can be preferably at least approximately 50% by weight and particularly preferably at least approximately 80% by weight. As a rule, it is advantageous if the propellant consists exclusively of HFA 134a and/or HFA 227 or their proportion in the total formulation is 90% by weight or more.

If desired, the aerosol formulations according to the invention can contain as a further propellant nitrogen or in particular dinitrogen monoxide (nitrous oxide) and/or carbon dioxide in an amount of approximately 0.0001 to 10% by weight. Concentrations of approximately 0.01 to 3% by weight are in general preferred and concentrations of approximately 0.1 to 1.0% by weight are particularly preferred; higher concentrations are as a rule only useful if the formulation contains a comparatively high proportion of cosolvent. As was found in WO-A-98/34595 and WO-A-00/06121, in fact propellants having more advantageous properties can be obtained if a small amount of dinitrogen monoxide and/or carbon dioxide is added to the customary propellants, in particular the hydrofluoroalkanes mentioned. Propellant mixtures of this type show—unlike dinitrogen monoxide and carbon dioxide as exclusive propellants—on increasing emptying only a slight decrease in the internal pressure in the container, which makes possible their use as propellants for metered-dose aerosols. Moreover, it was observed that the addition of dinitrogen monoxide and/or carbon dioxide facilitates the suspension of pharmaceutical active compounds, whereby it is more likely that the addition of surface-active substances and/or cosolvents can be abandoned or at least their proportion can be lowered. In addition, it was found that by addition of dinitrogen monoxide and/or carbon dioxide the undesired deposition of active compound in the oropharynx can be reduced and simultaneously the fine particle dose can be increased. Further, by addition of these propellants oxygen can be displaced from the hydrofluoroalkanes or other propellants, which improves the storage stability of oxidation-sensitive active compounds, and depending on the amount of dinitrogen monoxide and/or carbon dioxide, the internal pressure in the aerosol container can be adjusted such as is most useful for the respective application.

At 20° C., the aerosol formulations according to the invention preferably have a pressure of approximately 3 to 10 bar, in particular approximately 3.5 to 6 bar. If need be, a lower pressure can preferably be correspondingly increased by addition of dinitrogen monoxide and/or carbon dioxide.

The present invention as a rule allows the complete abandonment of cosolvents and conventional surface-active agents which are soluble in the propellant or propellant/cosolvent mixture. In particular, the aerosol formulation according to the invention can be essentially free of surface-active agents which are soluble, i.e. completely dissolved, in the propellant or propellant/cosolvent mixture, the expression “essentially free” preferably meaning a content of less than 0.0001% by weight, based on the total formulation. If desired, however, the further use of customary surface-active agents, such as oleic acid, lecithin, sorbitan trioleate and the like, is not excluded.

The addition of a small amount of cosolvent, however, can occasionally be advantageous. Suitable cosolvents are, for example, water, alcohols having 1 to 3 carbon atoms, alkanes having 3 to 6 carbon atoms, dialkyl ethers having 2 to 4 carbon atoms and the like. Examples of suitable cosolvents are: ethanol, propanol, isopropanol, ethylene glycol, propylene glycol, glycerol, propane, butane, isobutane, pentane, dimethyl ether and diethyl ether, with ethanol, ethylene glycol, glycerol, propylene glycol and diethyl ether or their mixtures and in particular ethanol as a rule being preferred. In general, however, the proportion of cosolvents such as ethanol, if present, is not above approximately 15% by weight, for example in the range from approximately 0.1 to 15% by weight, but preferably not above approximately 10% by weight and usually not above approximately 5% by weight, based on the total formulation.

Furthermore, the aerosol formulations according to the invention can if desired contain buffer substances or stabilizers such as citric acid, ascorbic acid, sodium EDTA, vitamin E, N-acetylcysteine and the like. In general, such substances, if present, are used in amounts of not more than approximately 1% by weight, for example in an amount of approximately 0.0001 to 1% by weight, based on the total formulation.

In general, however, aerosol formulations are preferred which consist of the abovementioned components (a), (b) and (c) or additionally contain ethanol as a cosolvent and/or additionally contain dinitrogen monoxide and/or carbon dioxide as a further propellant. A preferred aspect of the invention therefore relates to medical aerosol formulations, consisting of

(a) a pressure-liquefied, nontoxic propellant, selected from 1,1,1,2-tetrafluoroethane, 1,1,1,2,3,3,3-hepta-fluoropropane and mixtures thereof,

(b) an efficacious amount of at least one finely divided pharmaceutically active compound suspended in the propellant, selected from formoterol, salmeterol, fenoterol, clenbuterol, levalbuterol, ipratropium, oxytropium, glycopyrronium, tiotropium, budesonide, ciclesonide, mometasone, fluticasone, beclomethasone, flunisolide, loteprednol, triamcinolone, amiloride, rofleponide, salbutamol, terbutaline and pharmaceutically acceptable salts and derivatives thereof,

(c) a solid excipient, selected from calcium, magnesium and zinc salts of palmitic and stearic acid,

(d) optionally dinitrogen monoxide and/or carbon dioxide in an amount of from 0.0001 to 10% by weight, preferably 0.01 to 3% by weight, based on the total formulation, and

(e) optionally ethanol.

According to a preferred aspect, this formulation can contain as an active compound formoterol, salmeterol, fenoterol, clenbuterol, levalbuterol, ipratropium, oxytropium, glycopyrronium, tiotropium, budesonide, ciclesonide, mometasone, fluticasone, beclomethasone, flunisolide, loteprednol, triamcinolone, amiloride, rofleponide or a pharmaceutically acceptable salt or derivative of one of these active compounds, formulations of formoterol, salmeterol, fenoterol, levalbuterol, oxytropium, tiotropium, budesonide, mometasone, fluticasone and of pharmaceutically acceptable salts or derivatives of these active compounds being particularly preferred. According to a further preferred aspect, the formulation defined earlier can contain as active compound salbutamol, terbutaline or a pharmaceutically acceptable salt or derivative of one of these active compounds.

Examples of particularly preferred aerosol formulations according to the invention which can be mentioned are the following, in which the components in each case can be present in the amounts indicated above and in which, however, in particular the following components and amounts mentioned as preferred below have proven advantageous:

aerosol formulation, consisting of budesonide, at least one propellant selected from HFA 134a and HFA 227, at least one excipient, selected from calcium palmitate, calcium stearate, magnesium palmitate, magnesium stearate, zinc palmitate and zinc stearate, optionally an additional propellant, selected from dinitrogen monoxide and carbon dioxide, and optionally up to 0.5% by weight of ethanol; preferably, the formulation can consist of 0.1-1.0% by weight of budesonide, 0.005-0.2% by weight of excipient, 0-1% by weight of dinitrogen monoxide and/or carbon dioxide, 0-0.5% by weight of ethanol and of HFA 134a and/or HFA 227 (remainder); preferably the excipient can be magnesium stearate or a mixture of magnesium stearate and magnesium palmitate; the propellant is preferably HFA 134a or a mixture of HFA 134a and HFA 227; formulations which consist of budesonide, HFA 134a and excipient according to the invention, comprising magnesium stearate, are particularly preferred;

aerosol formulation, consisting of a beta-agonist, selected from formoterol, fenoterol, salbutamol, salmeterol, levalbuterol, terbutaline and pharmaceutically acceptable derivatives and salts thereof, at least one propellant, selected from HFA 134a and HFA 227, at least one excipient, selected from calcium palmitate, calcium stearate, magnesium palmitate, magnesium stearate, zinc palmitate and zinc stearate, optionally an additional propellant, selected from dinitrogen monoxide and carbon dioxide, and optionally ethanol; preferably the formulation can consist of 0.001-0.1% by weight of beta-agonist, 0.0001-0.2% by weight of excipient, 0-1% by weight of dinitrogen monoxide and/or carbon dioxide, 0.1-10% by weight of ethanol and of HFA 134a and/or HFA 227 (remainder); preferably the excipient can be magnesium stearate or a mixture of magnesium stearate and magnesium palmitate; the propellant is preferably HFA 227 or a mixture of HFA 134a and HFA 227; formulations are particularly preferred which contain as active compound formoterol or a pharmaceutically acceptable salt or derivative thereof, in particular formoterol fumarate or formoterol tartrate; likewise particularly preferred are formulations which as active compound contain salbutamol or a pharmaceutically acceptable salt or derivative thereof, in particular salbutamol sulfate or salbutamol acetate;

aerosol formulation, consisting of budesonide, a beta-agonist, selected from formoterol, fenoterol, salbutamol, salmeterol, levalbuterol, terbutaline and pharmaceutically acceptable derivatives and salts thereof, at least one propellant, selected from HFA 134a and HFA 227, at least one excipient, selected from calcium palmitate, calcium stearate, magnesium palmitate, magnesium stearate, zinc palmitate and zinc stearate, optionally an additional propellant, selected from dinitrogen monoxide and carbon dioxide, and optionally up to 0.5% by weight of ethanol; preferably the formulation can consist of 0.1-1.0% by weight of budesonide, 0.001-2% by weight (in particular 0.001-0.04% by weight) of beta-agonist, 0.005-0.2% by weight of excipient, 0-1% by weight of dinitrogen monoxide and/or carbon dioxide, 0-0.5% by weight of ethanol and of HFA 134a and/or HFA 227 (remainder); preferably the excipient can be magnesium stearate or a mixture of magnesium stearate and magnesium palmitate; preferably the formulation can be free of ethanol; formulations are particularly preferred in which the beta-agonist is formoterol or a pharmaceutically acceptable salt or derivative thereof, in particular formoterol fumarate or formoterol tartrate, and the propellant is HFA 134a or a mixture of HFA 134a and HFA 227, e.g. a mixture in the weight ratio of approximately 70:30;

aerosol formulation, consisting of fluticasone or a pharmaceutically acceptable salt or derivative (preferably fluticasone dipropionate) thereof, a beta-agonist, selected from formoterol, fenoterol, salbutamol, salmeterol, levalbuterol, terbutaline and pharmaceutically acceptable derivatives and salts thereof, at least one propellant, selected from HFA 134a and HFA 227, at least one excipient, selected from calcium palmitate, calcium stearate, magnesium palmitate, magnesium stearate, zinc palmitate and zinc stearate, optionally an additional propellant, selected from dinitrogen monoxide and carbon dioxide, and optionally up to 10% by weight of ethanol; preferably the formulation can consist of 0.1-1.0% by weight of fluticasone or salt or derivative thereof, 0.001-2% by weight (in particular 0.001-0.04% by weight) of beta-agonist, 0.005-0.2% by weight of excipient, 0-1% by weight of dinitrogen monoxide and/or carbon dioxide, 0.1-10% by weight of ethanol and of HFA 134a and/or HFA 227 (remainder); preferably the excipient can be magnesium stearate or a mixture of magnesium stearate and magnesium palmitate;

aerosol formulation, consisting of fluticasone or a pharmaceutically acceptable salt or derivative thereof (preferably fluticasone dipropionate), at least one propellant, selected from HFA 134a and HFA 227, at least one excipient, selected from calcium palmitate, calcium stearate, magnesium palmitate, magnesium stearate, zinc palmitate and zinc stearate, and optionally an additional propellant, selected from dinitrogen monoxide and carbon dioxide; preferably the formulation can consist of 0.1-1.0% by weight of fluticasone or its derivative, 0.005-0.5% by weight of excipient, 0-1% by weight (e.g. 0.1-1.0% by weight) of dinitrogen monoxide and/or carbon dioxide and of HFA 134a and/or HFA 227 (remainder); preferably the excipient can be zinc stearate or a mixture of zinc stearate and zinc palmitate; the propellant is preferably HFA 227 or a mixture of HFA 134a and HFA 227.

The preparation of the aerosol formulations according to the invention can be carried out in a manner known per se by introducing the micronized pharmaceutically active compound and the excipient into the pressure-liquefied propellant. The formulations can be prepared using customary stirrers and homogenizers. For filling, known processes such as the cold- or pressure-filling technique or modifications of these techniques can be employed. Suitable containers are, for example, pressure-safe containers made of glass, plastic or aluminum, which can be equipped with metering valves of, for example, 10 to 140 μl and can be provided with commercially available—also breath-triggered—mouth tube adapters.

The present invention thus makes possible the preparation of metered-dose aerosols having more advantageous properties, as is further illustrated with the aid of the following examples. In the examples, the expression “micronized” in each case means that the material concerned has a mean aerodynamic particle diameter of less than 6 μm.

### EXAMPLE 1

24.96 g of micronized budesonide and 3.12 g of magnesium stearate are weighed into a pressure batch vessel. After closing and evacuating the batch vessel, 7.8 kg of HFA 134a are added with stirring. After homogenization, the suspension obtained is filled into aluminum cans sealed with metering valves by means of pressure-filling technique.

The filled suspension is distinguished compared to a suspension prepared with identical amounts of budesonide and HFA 134a, but without magnesium stearate addition, by a greater flock volume and a longer suspension time of the suspended constituents. Using commercially available metering valves, the suspension according to the invention affords a better metering accuracy from stroke to stroke. Furthermore, the suspension according to the invention shows a markedly improved valve accessibility, while the valve in the comparison formulation without magnesium stearate is markedly more greatly stressed on activation (friction noises), which in the extreme case leads to leakiness in the valve.

### EXAMPLE 2

1.09 g of micronized formoterol fumarate and 0.182 g of magnesium stearate are weighed into a pressure batch vessel. After sealing and evacuating the batch vessel, 12.4 kg of HFA 227 are added, which had been treated with 0.4 kg of ethanol beforehand in another pressure batch vessel. After the homogenization of this mixture, the suspension obtained is filled into aluminum cans sealed with metering valves by means of pressure-filling technique.

### EXAMPLE 3

21.22 g of micronized budesonide and 0.54 g of magnesium stearate are weighed into a pressure batch vessel. After sealing and evacuating the batch vessel, 6.24 kg of a propellant mixture of HFA 227 and HFA 134a (weight ratio 30:70) are added, which have been treated beforehand with 0.002% by weight of ethanol in another pressure batch vessel. After the homogenization of this mixture, the suspension obtained is transferred to another pressure batch vessel, into which 0.64 g of formoterol fumarate has been weighed beforehand. The suspension is again homogenized and filled into aluminum cans sealed with metering valves by means of pressure filling technique.

### EXAMPLE 4

11.2 g of micronized glycopyrronium bromide and 1.1 g of magnesium stearate are weighed into a pressure batch vessel. After sealing and evacuating the batch vessel, 14 kg of a propellant mixture of HFA 227 and HFA 134a (weight ratio 50:50) are added with stirring, which has been treated beforehand with 1.4% by weight of ethanol in another pressure batch vessel. After the homogenization, the suspension obtained is filled into aluminum cans sealed with metering valves by means of pressure-filling technique.

### EXAMPLE 5

32 g of micronized fluticasone dipropionate and 3.9 g of zinc stearate are weighed into a pressure batch vessel. After sealing and evacuating the batch vessel, 9.75 kg of HFA 227 are added with stirring, which has been aerated beforehand with dinitrogen oxide in another pressure batch vessel and adjusted to a pressure of 5 bar at 20° C. After the homogenization, the suspension obtained is filled into aluminum cans sealed with metering valves by means of pressure-filling technique.

### EXAMPLE 6

14.4 g of micronized ipratropium bromide and 21.6 g of calcium stearate are weighed into a pressure batch vessel. After sealing and evacuating the batch vessel, 50.4 kg of HFA 227 are added with stirring, which has been aerated beforehand with dinitrogen oxide in another pressure batch vessel and adjusted to a pressure of 5 bar at 20° C. After the homogenization, the suspension obtained is filled into aluminum cans sealed with metering valves by means of pressure-filling technique.

1. A medical aerosol formulation for inhalation, comprising a pressure-liquefied, nontoxic propellant of the general formula
CₓH_{y}F_{z} (I)
in which x is the number 1, 2 or 3, y and z are each an integer ≧1 and y+z=2x+2,
an efficacious amount of a finely divided pharmaceutically active compound suspended in the propellant and a solid excipient, selected from calcium, magnesium and zinc salts of palmitic and stearic acid.

2. The aerosol formulation as claimed in claim 1, in which the propellant comprises 1,1,1,2-tetrafluoro-ethane, 1,1,1,2,3,3,3-heptafluoropropane or a mixture of the two.

3. The aerosol formulation as claimed in claim 1 or 2, comprising
(a) a pressure-liquefied, nontoxic propellant, selected from 1,1,1,2-tetrafluoroethane, 1,1,1,2,3,3,3-hepta-fluoropropane and mixtures thereof,
(b) an efficacious amount of a finely divided pharmaceutically active compound suspended in the propellant, selected from formoterol, salmeterol, fenoterol, clenbuterol, levalbuterol, ipratropium, oxytropium, glycopyrronium, tiotropium, budesonide, ciclesonide, mometasone, fluticasone, beclomethasone, flunisolide, loteprednol, triamcinolone, amiloride, rofleponide, salbutamol, terbutaline and pharmaceutically acceptable salts and derivatives thereof, and
(c) a solid excipient, selected from calcium, magnesium and zinc salts of palmitic and stearic acid.

4. The aerosol formulations as claimed in one of claims 1 to 3, consisting of
(a) a pressure-liquefied, nontoxic propellant, selected from 1,1,1,2-tetrafluoroethane, 1,1,1,2,3,3,3-hepta-fluoropropane and mixtures thereof,
(b) an efficacious amount of at least one finely divided pharmaceutically active compound suspended in the propellant, selected from formoterol, salmeterol, fenoterol, clenbuterol, levalbuterol, ipratropium, oxytropium, glycopyrronium, tiotropium, budesonide, ciclesonide, mometasone, fluticasone, beclomethasone, flunisolide, loteprednol, triamcinolone, amiloride, rofleponide, salbutamol, terbutaline and pharmaceutically acceptable salts and derivatives thereof, and
(c) a solid excipient, selected from calcium, magnesium and zinc salts of palmitic and stearic acid,
(d) optionally an additional propellant, selected from dinitrogen monoxide and carbon dioxide, in an amount of from 0.0001 to 10% by weight, based on the total formulation, and
(e) optionally ethanol.

5. The aerosol formulation as claimed in one of claims 2 to 4, in which 1,1,1,2-tetrafluoroethane, 1,1,1,2,3,3,3-heptafluoropropane or a mixture of the two is present in an amount of at least 50% by weight, based on the total formulation.

6. The aerosol formulation as claimed in one of claims 2 to 5, in which 1,1,1,2-tetrafluoroethane, 1,1,1,2,3,3,3-heptafluoropropane or a mixture of the two is present in an amount of at least 80% by weight, based on the total formulation.

7. The aerosol formulation as claimed in one of claims 1 to 6, in which the excipient is present in an amount of from 0.0001 to 1% by weight, based on the total formulation.

8. The aerosol formulation as claimed in one of claims 1 to 7, in which the excipient is present in an amount of 0.005 to 0.5% by weight, based on the total formulation.

9. The aerosol formulation as claimed in one of claims 1 to 8, in which the excipient is present in an amount of 0.01 to 0.2% by weight, based on the total formulation.

10. The aerosol formulation as claimed in one of claims 1 to 9, in which the suspended pharmaceutical active compound is present in an amount of from 0.0001 to 5% by weight, based on the total formulation.

11. The aerosol formulation as claimed in one of claims 1 to 10, in which the suspended pharmaceutically active compound is present in an amount of from 0.001 to 2% by weight, based on the total formulation.

12. The aerosol formulation as claimed in one of claims 1 to 11, in which the suspended pharmaceutically active compound and the excipient are present in a weight ratio of 50:1 to 1:10.

13. The aerosol formulation as claimed in one of claims 1 to 12, in which the suspended pharmaceutically active compound and the excipient are present in a weight ratio of 10:1 to 1:5.

14. The aerosol formulation as claimed in one of claims 1 to 13, in which the suspended pharmaceutically active compound has a mean aerodynamic particle diameter in the range from 1 to 6 μm.

15. The aerosol formulation as claimed in one of claims 1 to 14, in which the suspended pharmaceutically active compound is selected from formoterol, salmeterol, fenoterol, levalbuterol, oxytropium, tiotropium, budesonide, mometasone, fluticasone, salbutamol, terbutaline and pharmaceutically acceptable salts and derivatives thereof.

16. The aerosol formulation as claimed in one of claims 1 to 15, characterized in that it has a pressure of 3 to 10 bar at 20° C.

17. The aerosol formulation as claimed in one of claims 1 to 16, characterized in that it is essentially free of completely dissolved surface-active agents.

18. The aerosol formulation as claimed in one of claims 1 to 17, characterized in that it contains ethanol in an amount of from 0.1 to 15% by weight, based on the total formulation.

19. The aerosol formulation as claimed in one of claims 1 to 17, characterized in that it contains no ethanol.

20. The aerosol formulation as claimed in one of claims 1 to 17, consisting of
(a) a pressure-liquefied, nontoxic propellant, selected from 1,1,1,2-tetrafluoroethane, 1,1,1,2,3,3,3-hepta-fluoropropane and mixtures thereof,
(b) an efficacious amount of budesonide,
(c) a solid exdipient, selected from calcium palmitate, calcium stearate, magnesium palmitate, magnesium stearate, zinc palmitate and zinc stearate,
(d) optionally an additional propellant, selected from dinitrogen monoxide and carbon dioxide, in an amount of from 0.0001 to 10% by weight, based on the total formulation, and
(e) optionally ethanol in an amount of up to 0.5% by weight, based on the total formulation.

21. The aerosol formulation as claimed in claim 20, characterized in that budesonide is present in an amount of from 0.1 to 1% by weight and the excipient is present in an amount of from 0.005 to 0.2% by weight, in each case based on the total formulation.

22. The aerosol formulation as claimed in claim 20 or 21, in which the excipient comprises magnesium stearate.

23. The aerosol formulation as claimed in one of claims 20 to 22, characterized in that it contains no ethanol.

24. The aerosol formulation as claimed in one of claims 1 to 17, consisting of
(a) a pressure-liquefied, nontoxic propellant, selected from 1,1,1,2-tetrafluoroethane, 1,1,1,2,3,3,3-hepta-fluoropropane and mixtures thereof,
(b) an efficacious amount of a beta-agonist, selected from formoterol, fenoterol, salbutamol, salmeterol, levalbuterol, terbutaline and pharmaceutically acceptable salts and derivatives thereof,
(c) a solid excipient, selected from calcium palmitate, calcium stearate, magnesium palmitate, magnesium stearate, zinc palmitate and zinc stearate,
(d) optionally an additional propellant, selected from dinitrogen monoxide and carbon dioxide, in an amount of from 0.0001 to 10% by weight, based on the total formulation, and
(e) optionally ethanol.

25. The aerosol formulation as claimed in claim 24, characterized in that the beta-agonist is present in an amount of from 0.001 to 0.1% by weight and the excipient is present in an amount of from 0.0001 to 0.2% by weight, in each case based on the total formulation.

26. The aerosol formulation as claimed in claim 24 or 25, in which the excipient is magnesium stearate.

27. The aerosol formulation as claimed in one of claims 24 to 26, characterized in that it contains ethanol in an amount of from 0.1 to 10% by weight, based on the total formulation.

28. The aerosol formulation as claimed in one of claims 24 to 27, in which the beta-agonist is formoterol, formoterol fumarate or formoterol tartrate.

29. The aerosol formulation as claimed in one of claims 24 to 27, in which the beta-agonist is salbutamol, salbutamol sulfate or salbutamol acetate.

30. The-aerosol formulation as claimed in one of claims 1 to 17, consisting of
(a) a pressure-liquefied, nontoxic propellant, selected from 1,1,1,2-tetrafluoroethane, 1,1,1,2,3,3,3-hepta-fluoropropane and mixtures thereof,
(b) an efficacious amount of fluticasone or of a pharmaceutically acceptable salt or derivative thereof,
(c) a solid excipient, selected from calcium palmitate, calcium stearate, magnesium palmitate, magnesium stearate, zinc palmitate and zinc stearate, and
(d) optionally an additional propellant, selected from dinitrogen monoxide and carbon dioxide, in an amount of from 0.0001 to 10% by weight, based on the total formulation.

31. The aerosol formulation as claimed in claim 30, characterized in that fluticasone or its salt or derivative is present in an amount of from 0.1 to 1% by weight and the excipient is present in an amount of from 0.005 to 0.5% by weight, in each case based on the total formulation.

32. The aerosol formulation as claimed in claim 30 or 31, in which the excipient comprises zinc stearate.

33. The aerosol formulation as claimed in one of claims 1 to 17, in which the suspended pharmaceutically active compound is a beta-agonist, selected from formoterol, fenoterol, salbutamol, salmeterol, levalbuterol, terbutaline and pharmaceutically acceptable salts and derivatives thereof, and the formulation contains a further pharmaceutically active compound, selected from fluticasone, ipratropium, oxytropium, glycopyrronium, tiotropium, budesonide, mometasone, ciclesonide, rofleponide and pharmaceutically acceptable salts and derivatives thereof.

34. The aerosol formulation as claimed in claim 33, consisting of
(a) a pressure-liquefied, nontoxic propellant, selected from 1,1,1,2-tetrafluoroethane, 1,1,1,2,3,3,3-hepta-fluoropropane and mixtures thereof,
(b) an efficacious amount of budesonide and an efficacious amount of a beta-agonist, selected from formoterol, fenoterol, salbutamol, salmeterol, levalbuterol, terbutaline and pharmaceutically acceptable salts and derivatives thereof,
(c) a solid excipient, selected from calcium palmitate, calcium stearate, magnesium palmitate, magnesium stearate, zinc palmitate and zinc stearate,
(d) optionally an additional propellant, selected from dinitrogen monoxide and carbon dioxide, in an amount of from 0.0001 to 10% by weight, based on the total formulation, and
(e) optionally ethanol.

35. The aerosol formulation as claimed in claim 34, characterized in that budesonide is present in an amount of from 0.1 to 1% by weight, the beta-agonist is present in an amount of from 0.001 to 2% by weight and the excipient is present in an amount of from 0.005 to 0.2% by weight, the amounts in each case being based on the total formulation.

36. The aerosol formulation as claimed in claim 34 or 35, in which the excipient comprises magnesium stearate.

37. The aerosol formulation as claimed in one of claims 34 to 36, characterized in that it contains no ethanol.

38. The aerosol formulation as claimed in one of claims 34 to 37, in which the beta-agonist is formoterol, formoterol fumarate or formoterol tartrate.

39. The aerosol formulation as claimed in claim 33, consisting of
(a) a pressure-liquefied, nontoxic propellant, selected from 1,1,1,2-tetrafluoroethane, 1,1,1,2,3,3,3-hepta-fluoropropane and mixtures thereof,
(b) an efficacious amount of fluticasone or of a pharmaceutically acceptable salt or derivative thereof and an efficacious amount of a beta-agonist, selected from formoterol, fenoterol, salbutamol, salmeterol, levalbuterol, terbutaline and pharmaceutically acceptable salts and derivatives thereof,
(c) a solid excipient, selected from calcium palmitate, calcium stearate, magnesium palmitate, magnesium stearate, zinc palmitate and zinc stearate,
(d) optionally an additional propellant, selected from dinitrogen monoxide and carbon dioxide, in an amount of from 0.0001 to 10% by weight, based on the total formulation, and
(e) optionally ethanol in an amount of up to 0.5% by weight, based on the total formulation.

40. The aerosol formulation as claimed in claim 39, characterized in that fluticasone or its salt or derivative is present in an amount of from 0.1 to 1% by weight, the beta-agonist is present in an amount of from 0.001 to 2% by weight and the excipient is present in an amount of from 0.005 to 0.2% by weight, the amounts in each case being based on the total formulation.

41. The aerosol formulation as claimed in claim 39 or 40, in which the excipient comprises magnesium stearate.

42. The aerosol formulation as claimed in one of claims 39 to 41, characterized in that it contains ethanol in an amount of from 0.1 to 10% by weight, based on the total formulation.

43. The aerosol formulation as claimed in one of claims 1 to 42, containing 0.01 to 3% by weight of dinitrogen monoxide and/or carbon dioxide as an additional propellant.

44. The aerosol formulation as claimed in one of claims 1 to 43, containing 0.1 to 1% by weight of dinitrogen monoxide and/or carbon dioxide as an additional propellant.

45. The aerosol formulation as claimed in one of claims 1 to 42, characterized in that it contains no further propellant in addition to 1,1,1,2-tetrafluoroethane, 1,1,1,2,3,3,3-heptafluoropropane or mixtures thereof.

46. A compressed gas pack, comprising a medical aerosol formulation, as defined in one of claims 1 to 45, in a pressure-tight container provided with a metering valve.

46. A process for the production of a medical aerosol formulation, as defined in claim 1, characterized in that the pharmaceutically active compound and the excipient are introduced into the pressure-liquefied, nontoxic propellant.

47. The use of a carboxylic acid salt, selected from calcium, magnesium and zinc salts of palmitic and stearic acid, as a solid excipient in medical suspension aerosol formulations for inhalation, comprising a pressure-liquefied, nontoxic propellant of the general formula
CₓH_{y}F_{z} (I)
in which x is the number 1, 2 or 3, y and z are each an integer ≧1 and y+z=2x+2,
and a finely dispersed pharmaceutically active compound suspended in the propellant.

48. The use as claimed in claim 47 for the purpose of improving the suspension stability.

49. The use as claimed in claim 47 for the purpose of improving the metering accuracy.

50. The use as claimed in claim 47 for the purpose of improving the valve function of metering valves.

51. The use as claimed in claim 47 for the purpose of improving the chemical stability of the pharmaceutically active compound.

52. The use as claimed in claim 51 for the purpose of improving the moisture resistance of the pharmaceutically active compound.
